(19) United States
(12) Patent Application Publication
(10) Pub. No.: US 2016/0374931 A1
(43) Pub. Date: Dec. 29, 2016

(54) Method For Forming Particles With Cross-Linked Coatings For Cosmetic Applications

(71) Applicant: ELC Management LLC, Melville, NY (US)

(72) Inventors: Raffi Balian, Bay Shore, NY (US); Timothy Gray, New York, NY (US); Peter Tsolis, South Huntington, NY (US)

(21) Appl. No.: 15/183,911

(22) Filed: Jun. 16, 2016

Related U.S. Application Data
(60) Provisional application No. 62/186,048, filed on Jun. 29, 2015.

(51) Int. Cl. A61K 8/895; A61Q 1/02; A61K 8/27; A61K 8/02; C09D 143/04; A61K 8/29

(52) U.S. Cl. CPC A61K 8/895; C09D 143/04; A61K 8/29; A61K 8/27; A61K 8/0233; A61Q 1/02; A61K 2800/43

(57) ABSTRACT

The present invention relates to a method for forming coated particles, comprising the steps of: agitating a volume of core particles while heating to a constant average temperature of about 40° C. Once particles are generally evenly mixed, with no apparent agglomeration, the vinyl-functional silicone polymer, hydride-functional crosslinker, and metal catalyst are combined in a premix under continuous propeller-mixing. The vinyl-functional silicone polymer, hydride-functional crosslinker, and metal catalyst premix is then siphoned into the main vessel, containing the heated core particles, at rate that ensures no clumping or agglomeration of the particulates. Once even coating has been achieved, the main vessel is heated to an average temperature of about 80° C., then cooled back down to room temperature (about 20° C.-28° C.). The coated material is then passed through a 45 μm sieve to remove any large particulates that may be present.

### FIELD OF THE INVENTION

The present invention relates to topical compositions comprising coated particles for preventing bleeding of colored particles and/or providing particle stability in a solvent system, as well as methods of making the same.

### BACKGROUND OF THE INVENTION

Cosmetic or topical compositions typically comprise one or more particulate components, such as, for example, pigments or dyes, fillers, thickeners, sunscreen agents, and the like. Such particulate components are often insoluble in the respective solvent or carrier system and if so remain dispersed or suspended in the cosmetic or topical compositions.

The menu of suitable particulate components is limited by factors such as stability, incompatibility with other cosmetic components, skin irritability, reactivity, and the like. In the case of color cosmetics, many pigments and/or dyes which are soluble in oils or polar solvents, such as water, may suffer from “bleeding” into the broader composition.

Furthermore, whenever there are changes in the pH and temperature in the surrounding environment, dispersed or suspended particles may agglomerate with one another and precipitate out of the composition. The smaller the particle size, the larger the active surface area, and the more susceptible such particulate components are toward adverse interactions or interference with other ingredients or components in the cosmetic or topical compositions, which may destabilize the cosmetic or topical compositions or reduce the overall performance thereof.

Coatings for particles are well known, but they typically suffer from one or more drawbacks such as permeability, opacity, solubility, and agglomeration.

Accordingly, there is a continuing need for treating or modifying the cosmetic particles in order to eliminate or mitigate the above-described drawbacks. There is also a need to improve the overall stability and chromatic appearance of particles in cosmetic compositions without adversely affecting the chemical and physical properties of the particles.

### SUMMARY OF THE INVENTION

In one aspect, the present invention relates to a topical composition comprising a dispersion of coated particles in a cosmetically or pharmaceutically acceptable carrier, wherein one or more core particles comprise a heat-curable coating which is the reaction product of a vinyl-functional silicone polymer, a hydride-functional crosslinker, and a metal catalyst.

In another aspect, the present invention relates to a method for forming coated particles, comprising the steps of:

1) Agitating a volume of core particles while heating to a constant average temperature of about 40° C.

2) Once particles are generally evenly mixed, with no apparent agglomeration, the vinyl-functional silicone polymer, hydride-functional crosslinker, and metal catalyst are combined in a premix under continuous propeller-mixing.

3) The vinyl-functional silicone polymer, hydride-functional crosslinker, and metal catalyst premix is then siphoned into the main vessel, containing the heated core particles, at rate that ensures no clumping or agglomeration of the particulates.

4) Once even coating has been achieved, the main vessel is heated to an average temperature of about 80° C., then cooled back down to room temperature (about 20° C.-28° C.).

5) The coated material is then passed through a 45 μm sieve to remove any large particulates that may be present.

The final coated particles are generally referred to herein as a “metal catalyzed thermal cure”. As will be discussed, Platinum (Pt) is a particularly preferred catalyst. Therefore, the coated particles may also be referred to as a “Pt catalyzed thermal cure”. However, such references should not be construed as limiting.

Other aspects and objectives of the present invention will become more apparent from the ensuing description, examples, and claims.

### DETAILED DESCRIPTION OF THE INVENTION

### Vinyl Silicone Coating

The compositions herein comprise particles which are coated with a heat curable silicone composition.

The vinyl silicone coatings herein contain vinyl-containing polydiorganosiloxane base polymers which are generally comprised of siloxane units having substituent groups including lower alkyl radicals having up to about 8 carbon atoms such as methyl, ethyl, propyl, isopropyl, etc., and monovalent alkenyl radicals of from about 2 to 20 carbon atoms such as vinyl, allyl, butenyl, etc., including cycloalkenyl. These polymers are prepared by methods known in the art and are commercially available with varying degrees of functionality. They generally have a viscosity ranging from about 50 to about 100,000 centipoise 25 C.

The vinyl silicone coating also comprises a SiH-containing polysiloxane for use as a crosslinking agent. The curing reaction which takes place between the vinyl-functional polysiloxane base polymer and the SiH-containing crosslinking agent is an addition cure reaction, known as hydrosilation. The coatings may be thermally cured by means of a platinum group metal catalyzed crosslinking reaction between the vinyl groups of the base polymer and the SiH reaction sites of the crosslinker.

Exemplary polydiorganosiloxane base polymers and SiH-containing polysiloxanes are provided in U.S. Pat. No. 5,223,344.

Suitable hydrosilation catalysts are known and include platinum group metal catalysts utilizing such precious metals as ruthenium, rhodium, palladium, osmium, iridium and platinum, and complexes of these metals. Preferred are platinum metal catalysts. Examples of such hydrosilation catalysts are described in, inter alia, Lamoreaux, U.S. Pat. No. 3,220,972; Karstedt, U.S. Pat. Nos. 3,715,334; 3,775,452 and 3,814,730; Ashby, U.S. Pat. Nos. 4,421,903; and 4,288,345; and Saruyama et al., U.S. Pat. No. 5,057,476.

The selection of the particular catalyst will depend upon such factors as speed of reaction desired, expense, useful shelf-life, useful pot-life and the temperature at which the cure reaction is to take place. The amount of catalyst employed is not critical, an long as proper crosslinking is achieved; however, as indicated above, the high cost of these precious metal catalysts makes their conservative use obligatory. As with any catalyst, it is preferable to use the smallest effective amount possible, for the coating compositions described herein, enough total catalyst is used to provide from about 5 to about 500 parts per million of precious metal as precious metal.

In one embodiment, the coatings may include a silane cure accelerator additive. The silane additives of the present invention are those having at least two hydrogen atoms bonded to the silicon atom. Preferred are those having the general formula R²₂SiH₂ and R²SiH₃ where R² is selected from the group consisting of alkyl radicals; halides such as chlorine, bromine and iodine; cycloalkyl radicals and phenyl radicals.

Typically the silane cure accelerator additives are selected from silanes such as, but not limited to, dichlorosilane, dimethylsilane, diethylsilane, dipropylsilane, dibutylsilane, dipentylsilane, dihexylsilane, diheptylsilane, dioctylsilane, dinonylsilane, didecylsilane, dicyclopentylsilane, dicyclohexylsilane, dicycloheptylsilane, diphenylsilane, phenylchlorosilane, phenylethylsilane, methyloctylsilane, methyldecylsilane, phenylmethylsilane, phenylethylsilane, phenylcyclohexylsilane and methylcycloheptylsilane. Preferred are diphenylsilane, phenylsilane, dioctylsilane, methyldecylsilane, phenylmethylsilane and didecylsilane. Most preferred is diphenylsilane and phenylsilane.

The silane cure accelerator additives are employed in small effective amounts. Typically the silane cure accelerator additive is present in the compositions of the present invention in amounts ranging from about 0.25 to about 20 moles of silane to moles of catalyst metal, most preferably from about 0.5 to about 5 moles of silane to moles of catalyst metal.

The compositions may also comprise an inhibiting agent. These serve to prevent premature cure in one-package systems and also extend the shelf-life of stored products and the pot-life of products in use. That is, at room temperature, the complete silicone composition will not gel prematurely, as often happens with catalyzed silicone compositions. Especially useful are dialkyl and dialkenylcarboxylic ester inhibitors such as diallylmaleate and dimethylmaleate. These are known to those skilled in the art and are described in U.S. Pat. No. 4,256,870.

Additional inhibitors include but are not limited to acetylenic alcohols such as those described in U.S. Pat. No. 3,445,420; amines such as those described in U.S. Pat. No. 4,584,361; isocyanurates such as those described in U.S. Pat. No. 3,882,083; ene-ynes such as those described in U.S. Pat. No. 4,465,818; vinyl acetates such as those described in U.S. Pat. No. 4,476,166; and acetylene dicarboxylates such as those described in U.S. Pat. No. 4,347,346.

Additional ingredients may be added to the compositions to lend specific properties and allow the compositions to be tailored to an end-user's needs. For example, the compositions may be dispersed in a solvent or used in a 100% solids formulation, as required.

Vinyl gum cure accelerators, such as those described in Eckberg, U.S. Pat. No. Re. 31,727, can be added to the compositions of the present invention. Adding from about 0.5 to about 10 weight percent based on the total weight of the base polymer of the vinyl gum reduces the required curing time.

Other conventional additives, such as controlled release additives (“CRAs”), anti-microbial agents, anti-foaming agents and other additives familiar to persons skilled in the art are also contemplated by the present disclosure.

Fillers and additives to improve the thermal stability, weather resistance, chemical resistance, flame retardancy or mechanical strength or to reduce the degree of gas permeation can also be added. Examples of these fillers and additives include fumed silica, quartz powder, glass fiber, carbon black, alumina, metal oxides such as iron oxide and titanium oxide and metal carbonates such as calcium carbonate and magnesium carbonate. Moreover, suitable pigments, dyes, blowing agents or antioxidants can be added within a range which does not interfere with the curing process.

### Vinyl Silicone Coating Method

Methods for coating particulate materials are well known. However, the Pt catalyzed thermal cure herein is formed with a coating material which is generally applied to large, generally flat, surfaces. The present invention has adapted the heat-curing method for coating large surfaces to enable coating particulates having an average particle size of less than about 45 μm. The steps for forming the Pt catalyzed thermal cure are provided as follows:

1) Agitating a volume of core particles while heating to a constant average temperature of about 40° C. A conventional maximum volume for industrial applications is about 22 Liters in, for example, a Littleford Model M-5 Laboratory Mixer.

2) Once particles are generally evenly mixed, with no significant agglomeration, the vinyl-functional silicone polymer, hydride-functional crosslinker, and platinum catalyst are combined in a premix under continuous propeller-mixing.

3) The vinyl-functional silicone polymer, hydride-functional crosslinker, and platinum catalyst premix is then siphoned into the main vessel, containing the heated core particles, at rate that ensures no clumping or agglomeration of the particulates.

4) Once even coating has been achieved, the main vessel is heated to an average temperature of about 80° C., then cooled back down to room temperature (about 20° C.-28° C.).

5) The coated material is then passed through a 45 μm sieve to remove any large particulates that may be present.

The coated particulate material resulting from the process hereinbefore is referred to as a “Pt catalyzed thermal cure”. The Pt catalyzed thermal cure is particularly useful for sealing particulate surfaces via its crosslinked coating. As such, a broad variety of cosmetic particulate materials, which would otherwise “bleed” into the cosmetic composition, are enabled for use in cosmetic formulas. Conventional core particles, and those which are otherwise prone to bleeding, are therefore useful as components of the Pt catalyzed thermal cure. And such exemplary core particles are described, in detail, hereinafter.

### Cosmetic Core Particles

The present invention provides coated particulate components that are useful in cosmetic or topical compositions, as well as methods for making such coated particulate components. Specifically, the coated particles each comprises at least one core particle, which is preferably a solid particle that is insoluble in the respective solvent system (either aqueous or anhydrous), coated by the vinyl silicone release coating herein.

The core particles useful for the present invention can be any particulate components that are commonly used in cosmetic or pharmaceutical compositions, which include, but are not limited to mineral pigments and fillers such as, for example, talc, kaolin, mica, bismuth oxychloride, chromium hydroxide, barium sulfate, polymethylmethacrylates (PMMA), boron nitride, nylon beads, polymeric powders (e.g., BPD 500 powders comprised of hexamethylene diisocyanate/trimethylol hexyllactone crosspolymer and silica that is commercially available from Kobo Products, Inc. at South Plainfield, N.J.), silica, silica beads, lakes (e.g., aluminum or calcium lake), metal oxides (e.g., black, yellow or blue iron oxide, chromium oxide, zinc oxide, and titanium dioxide), physical and chemical sunscreen agents, and any other organic and inorganic powders or particles.

In one embodiment, the core particles can be dyes or pigments. Preferably, the dyes or pigments may be water-soluble. In one particular embodiment, the pigments may be water- soluble biological pigments, such as anthocyanins or betalains.

The core particles can be of any regular or irregular shape, such as, for example, spherical, cubic, cylindrical, planar, fibrous, laminar, and the like. The average particle size of the core particles as used in the present invention is preferably from about 0.1 micron to about 50 microns, more preferably from about 0.25 micron to about 25 microns, and most preferably from about 0.45 micron to about 9 microns. The core particles preferably constitute about 10 to about 99 percent by total weight of the coated particles, and more preferably, about 40 to 90 percent by total weight of the coated particles.

### Dyestuffs

According to at least one embodiment of the present disclosure, the composition may comprise at least one dyestuff.

For the purposes of the present disclosure, the term “dyestuff” means a compound capable of producing a colored optical effect when it is formulated in sufficient amount in a suitable cosmetic medium.

In at least one embodiment, the at least one dyestuff is chosen from pigments, nacres, flakes, liposoluble dyes and water-soluble dyes, and mixtures thereof.

In the present disclosure, the term “pigments” should be understood as meaning white or colored, mineral or organic particles, which are insoluble in the liquid organic phase and which are intended to color and/or opacify the composition.

The term “nacres”, as used in the present disclosure, should be understood as meaning iridescent particles, produced, for instance, by certain molluscs in their shell or else synthesized, which are insoluble in the medium of the composition.

In the context of the present disclosure, the term “dyes” should be understood as meaning generally organic compounds that are soluble in fatty substances such as oils or in an aqueous phase.

In at least one embodiment, the at least one dyestuff is present in an amount ranging from 0.01% to 40% by weight, such as, for example, from 5% to 30% by weight or from 5% to 20% by weight, relative to the total weight of the composition.

According to at least one embodiment of the present disclosure, the at least one dyestuff comprises at least one pigment.

The at least one pigment may be chosen from mineral pigments, organic pigments and composite pigments (i.e. pigments based on mineral and/or organic materials).

In the present disclosure, the term “pigments” should be understood as meaning mineral or synthetic particles of any form, endowed with an optical effect, which are insoluble in the medium of the composition irrespective of the temperature at which the composition is manufactured.

The at least one pigment may be chosen from, for example, monochromatic pigments, lakes, nacres and pigments with an optical effect, for instance reflective pigments and goniochromatic pigments.

In at least one embodiment, the mineral pigments are chosen from metal oxide pigments, mica coated with titanium dioxide, mica coated with bismuth oxychloride, titanium mica coated with iron oxide, titanium mica coated with ferric blue, titanium mica coated with chromium oxide, iron oxides, titanium dioxide, zinc oxides, cerium oxide, zirconium oxide or chromium oxide; manganese violet, Prussian blue, ultramarine blue, ferric blue, bismuth oxychloride, colored nacreous pigments such as titanium mica with iron oxides, titanium mica with, for example, ferric blue or chromium oxide, titanium mica with an organic pigment of the abovementioned type, and also nacreous pigments based on bismuth oxychloride, and mixtures thereof.

Organic pigments may include, for example: cochineal carmine; organic pigments of azo dyes, anthraquinone dyes, indigoid dyes, xanthene dyes, pyrene dyes, quinoline dyes, triphenylmethane dyes or fluorane dyes; organic lakes or insoluble salts of sodium, potassium, calcium, barium, aluminium, zirconium, strontium or titanium, or of acidic dyes such as azo dyes, anthraquinone dyes, indigoid dyes, xanthene dyes, pyrene dyes, quinoline dyes, triphenylmethane dyes or fluorane dyes. These dyes may comprise at least one carboxylic or sulfonic acid group; and melanin pigments.

Among the organic pigments that may be used according to the present disclosure, non-limiting mention may be made of D&C Blue No 4, D&C Brown No 1,D&C Green No 5, D&C Green No 6, D&C Orange No 4, D&C Orange No 5, D&C Orange No 10, D&C Orange No 11, D&C Red No 6, D&C Red No 7, D&C Red No 17, D&C Red No 21, D&C Red No 22, D&C Red No 27, D&C Red No 28, D&C Red No 30, D&C Red No 31, D&C Red No 33, D&C Red No 34, D&C Red No 36, D&C Violet No 2, D&C Yellow No 7, D&C Yellow No 8, D&C Yellow No 10, D&C Yellow No 11, FD&C Blue No 1, FD&C Green No 3, FD&C Red No 40, FD&C Yellow No 5 and FD&C Yellow No 6.

According to at least one embodiment, the at least one pigment present in the composition according to the disclosure is chosen from hydrophobic-coated pigments.

In the context of the present disclosure, the term “hydrophobic-coated pigments” means pigments surface-treated with a hydrophobic agent to make them compatible with the fatty phase of the emulsion, in order that they may show good wettability with the oils of the fatty phase. These treated pigments may be well dispersed in the fatty phase.

The pigments intended to be coated may be mineral or organic pigments described above.

In at least one embodiment, iron oxide or titanium dioxide pigments are used.

The hydrophobic-treatment agent may be chosen from silicones, for instance methicones, dimethicones or perfluoroalkylsilanes; fatty acids, for instance stearic acid; metal soaps, for instance aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids may comprise an acyl group comprising from 8 to 22 carbon atoms, for instance a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

In the present disclosure, the term “alkyl” mentioned in the compounds mentioned above may denotes an alkyl group comprising from 1 to 30 carbon atoms, such as from 5 to 16 carbon atoms.

Hydrophobic-treated pigments are described, for example, in European Patent Application No. EP-A-1 086 683. While such pigments may be further coated by the coatings herein, the coatings of the present invention may be advantages over traditional hydrophobic coating treatments. For example many silicone coatings carry inherent coloration which may interfere with exhibition of the color of any core particle. But the coatings herein are substantially transparent and colorless, therefore transmitting color of the coated core particle with greater efficiency than hydrophbically-treated particles.

The pigments may be present in the composition according to at least one embodiment of the present disclosure in an amount ranging from 2% to 40% by weight, such as from 5% to 30% by weight or from 5% to 20% by weight, relative to the total weight of the composition.

Liposoluble dyes may be chosen from, for example, Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The water-soluble dyes may be chosen from, for example, beetroot juice, methylene blue and caramel. When formulated into topical compositions, the coated particles of the present invention provide various advantages and benefits that are not available in their un-encapsulated or “naked” counterparts. For example, because the core particles are sealed off by the coating layers from potentially destabilizing or degrading active ingredients in the topical composition, they are significantly more stable than their un-encapsulated or “naked” counterparts. Further, if the core particles contain material or materials potentially capable of cause generation of reactive oxygen species (ROS), which may in turn degrade or otherwise interfere with other active ingredients in the topical composition, the antioxidant coating layer functions to scavenge the ROS and thereby reduce the interference or degradation and improves the overall stability of the topical composition. Coating of the core particles by the water-insoluble metal salt of N-acylamino acid, which is hydrophobic, may also impart hydrophobicity to certain core particles that are intrinsically hydrophilic and allow such core particles to be formulated into oil or silicone phases that are typically incompatible with un- encapsulated or “naked” hydrophilic particles. It is important to note that the desired chemical and/or physical properties of the core particles should remain substantially unaffected by the presence of the coating layers described hereinabove.

The coated particles can be added directly to any pharmaceutically or cosmetically acceptable carrier to form a cosmetic or topical composition. For purpose of the present invention, pharmaceutically or cosmetically acceptable carriers are substances that are biologically compatible with human skin and can be used to formulate active ingredients described hereinabove and/or hereinafter into a cream, gel, emulsion, liquid, suspension, powder, foundation, nail coating, lip treatment, mascara, skin oil, or lotion that can be topically applied. In the case where the cosmetically acceptable carrier is in the form of an emulsion, it may contain from about 0.1 to 99%, preferably from about 0.5 to 95%, more preferably from about 1 to 80% by weight of the total composition of water and from about 0.1 to 99%, preferably from about 0.1 to 80%, more preferably from about 0.5 to 75% by weight of the total composition of oil. If the composition is anhydrous, it may comprise from about 0.1 to 90 wt % of oil and from about 0.1 to 75 wt % of other ingredients such as pigments, powders, non-aqueous solvents (such as mono-, di-, or polyhydric alcohols, etc. In the case where the composition is in the form of an aqueous based gel, solution, or suspension, it may comprise from about 0.1 to 99 wt % of water and from about 0.1 to 75 wt % of other ingredients such as botanicals, non-aqueous solvents, etc.

The pharmaceutically or cosmetically acceptable carrier or carriers can be present in the topical or cosmetic composition of the present invention at an amount ranging from about 0.1% to about 99.9%, preferably from about 5% to about 99.5%, more preferably from about 10% to about 99%, and most preferably from about 10% to 90% by total weight of the topical or cosmetic composition.

The topical or cosmetic composition may contain one or more skin care actives, which are agents that provide benefits to the skin, rather than merely improving the physical or aesthetic characteristics of the topical composition. If present, such skin care actives may range from about 0.01 to 50%, preferably from about 0.05 to 35% by weight of the total composition. Exemplary skin care additives that can be used in the topical or cosmetic compositions of the present invention include, but are not limited to: chemical or physical sunscreens, self-tanning agents such as dihydroxyacetone, anti-acne agents (e.g., resorcinol, salicylic acid, benzoyl peroxide, and the like), enzyme-inhibiting agents, collagen-stimulating agents, agents for the eradication of age spots and keratoses, analgesics, anesthetics, antimicrobials (e.g., antibacterials, antiyeast agents, antifungal agents, and antiviral agents), antidandruff agents, antidermatitis agents, antipruritic agents, antiemetics, anti-inflammatory agents, antihyperkeratolytic agents, antiperspirants, antipsoriatic agents, antiseborrheic agents, antihistamine agents, skin lightening agents, depigmenting agents, skin soothing/healing agents (e.g., aloe vera extract, allantoin, and the like), corticosteroids, hormones, proteins or peptides, vitamins and derivatives thereof (e.g., vitamin A, vitamin E, vitamin B₃, vitamin B₅, and the like), exfoliants, retinoids (e.g., retinoic acid and retinol), farnesol, bisabolol, phytantriol, glycerol, urea, guanidine (e.g., amino guanidine), clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, minocycline, hydroquinone, naproxen, ibuprofen, theophylline, cromolyn, albuterol, topical steroids (e.g., hydrocortisone, hydrocortisone 21-acetate, hydrocortisone 17-valerate, and hydrocortisone 17-butyrate), betamethasone valerate, betamethasone diproprionate, benzoyl peroxide, crotamiton, propranolol, promethazine, and mixtures or derivatives thereof. In a preferred, but not necessary embodiment of the present invention, the topical composition comprises one or more skin care actives selected from the group consisting of sunscreen agents, self-tanning agents, anti-aging agents, anti-wrinkle agents, anti-acne agents, antimicrobials, anti-inflammatory agents, skin-lightening agents, proteins or peptides, vitamins and derivatives thereof, exfoliants, ingredients that stimulate DNA repair, ingredients that provide immune protection, ingredients that stimulate cell renewal, ingredients that stimulate skin barrier repair, moisturizers, and mixtures thereof.

### Carrier

The cosmetically acceptable carrier may also contain one or more oils, which may be silicone, organic, or mixtures thereof. If present, such oils may range from about 0.1 to 99% by weight of the total composition and include volatile or non-volatile silicones such as cyclomethicone; methyl trimethicone; octamethyltrisiloxane; decamethyltetrasiloxane; dodecamethylpentasiloxane; dimethicone; phenyl trimethicone trimethylsiloxyphenyl dimethicone; phenyl dimethicone; cetyl dimethicone; dimethicone copolyol, cetyl dimethicone copolyol; glycerolated silicones such as lauryl PEG-9 polydimethylsiloxyethyl dimethicone; or mixtures thereof. Suitable esters include mono-, di-, or triesters of C4-30 fatty acids and mono-, di-, or polyhydric C1-20 alcohols, such as fatty acid (e.g., stearyl, behenyl, and isostearyl) esters of glycerin, or fatty acid esters of alpha hydroxyl acids such as citric, malic, or lactic acids and the like. Suitable hydrocarbons include monomeric or polymeric olefins or alpha olefins, such as polyisobutene, polydecene, polybutene, or hydrogenated derivatives thereof.

The cosmetically acceptable carrier may also comprise one or more humectants. If present, they may range from about 0.1 to 20% by weight of the total composition and include C1-4 alkylene glycols such as butylene, propylene, ethylene glycol, glycerin and the like.

The cosmetically acceptable carrier may also contain one or more waxes preferably having a melting point ranging from about 30 to 150° C. If present, such waxes may range from about 0.1 to 45% by weight of the total composition and include animal, vegetable, mineral, or silicone waxes. Examples include alkyl dimethicones stearyl dimethicone, candelilla, polyethylene, ozokerite, beeswax, and the like.

The cosmetically acceptable carrier may also comprise one or more organosiloxane elastomers, either emulsifying or non-emulsifying. If present, such elastomers may range from about 0.1 to 30% by weight of the total composition. Examples of suitable elastomers include dimethicone/vinyl dimethicone crosspolymer; dimethicone/dimethicone PEG/PPG 10/15 crosspolymer; and the like.

The cosmetically acceptable carrier may also include one or more pigments or powders or mixtures thereof. If present, the suggested ranges of such pigments or powders are from about 0.1 to 85% by weight of the total composition. The particle sizes of such pigments or powders may range from about 0.05 to 200microns but are preferably about 50-100 microns. Examples of pigments include organic pigments such as D&C or FD&C colors or Lakes thereof including blues, browns, reds, etc; or inorganic iron oxides such as brown, yellow, green, red, iron oxides. Suitable powders include titanium dioxide, nylon, PMMA, boron nitride, mica, and the like.

The cosmetically acceptable carrier may also comprise one or more nonionic surfactants, particularly if the topical or cosmetic composition of the present invention is provided in the emulsion form. If present, such surfactants may range from about 0.1 to 20% by weight of the total composition. Suitable surfactants include ethoxylated fatty C6-30 alcohols such as steareth, beheneth, ceteth where the number following each of the surfactants refers to the number of repeating ethylene oxide groups which may range from 2 to 250, e.g. steareth-2, beheth-30 and so on.

While the present invention has been described hereinabove with reference to specific embodiments, features and aspects, it will be recognized that the invention is not thus limited, but rather extends in utility to other modifications, variations, applications, and embodiments, and accordingly all such other modifications, variations, applications, and embodiments are to be regarded as being within the spirit and scope of the present invention.

What is claimed is:

1. A method for forming coated particles, comprising the steps of:
a) agitating a volume of core particles in a first vessel while heating said core particles to an average temperature of about 40° C.;
b) combining a vinyl-functional silicone polymer, hydride-functional crosslinker, and a metal catalyst in a second vessel to form a premix;
c) combining said core particles with said premix to form a mixture;
d) heating said mixture to an average temperature of about 80° C., then allowing said mixture to cool to ambient temperature.

2. A method according to claim 1, further comprising the step of separating particles having an average particle size of less than about 45 μm from said mixture.

3. A method according to claim 1, wherein said metal catalyst is selected from the group consisting of ruthenium, rhodium, palladium, osmium, iridium and platinum, and complexes and mixtures thereof.

4. A method of claim 1, wherein the core particle comprises titanium dioxide, zinc oxide, or a combination thereof.
